(19) United States
(12) Patent Application Publication
(10) Pub. No.: US 2005/0084413 A1
(43) Pub. Date: Apr. 21, 2005

(54) Artificial flower with electric fan and a fragrance source

(76) Inventor: Virgil E. Stanley III, Indianapolis, IN (US)

Correspondence Address: COATS & BENNETT, PLLC, P O BOX 5, RALEIGH, NC, 27602, US

(21) Appl. No.: 10/959,267

(22) Filed: Oct. 6, 2004

Related U.S. Application Data
(63) Continuation-in-part of application No. 10/756,224, filed on Jan. 13, 2004.
Application No. 10/756,224 is a continuation-in-part of application No. 10/164,818, filed on Jun. 7, 2002, now Pat. No. 6,830,733.

(30) Foreign Application Priority Data
Apr. 11, 2003 (WO) PCT/US03/11043

(51) Int. Cl. A61L 9/12; A61L 9/04

(52) U.S. Cl. 422/5; 422/124

(57) ABSTRACT

An artificial flower is provided that includes a series of hollow stems and a series of petals with the respective petals being connected to upper end portions of the respective hollow stems. Opposed terminal ends of the stems are connected to a series of outlets that form a part of a manifold. The manifold, in turn, overlies or is disposed above an electric squirrel cage-type fan having a housing and an air inlet and an air outlet. Secured or disposed adjacent the electric fan is a fragrance housing having a fragrant source disposed therein and including an air inlet and an air outlet. Air is induced into and through the fragrance housing producing an air-fragrance mixture. Upon leaving the fragrance housing, the air-fragrance mixture is directed through the fan and therefrom into the manifold where the air-fragrance is dispersed through the respective hollow stems, ultimately resulting in the air-fragrance mixture being dispersed adjacent the petals.

### CROSS REFERENCE TO RELATED APPLICATIONS

This is a continuation-in-part of U.S. patent application Ser. No. 10/756,224 filed Jan. 13, 2004 and entitled “Artificial Flower” which was, in turn, a continuation-in-part of U.S. patent application Ser. No. 10/164,818 filed on Jun. 7, 2002 and also entitled “Artificial Flower.”

### FIELD OF THE INVENTION

The present invention relates to artificial flowers, and more particularly to an artificial flower having a fragrance source and means for dispersing a fragrance from the source about the artificial flower.

### BACKGROUND OF THE INVENTION

Artificial flowers are known. Technologically it is possible to create an artificial flower that possesses a realistic appearance. Such artificial flowers include pleasing petals and even sometimes associated vegetation. However, there are many drawbacks and difficulties experienced in providing a cost effective and marketable artificial flower. For the most part, past designs have tended to be expensive and difficult to manufacture at an appropriate price point. Further, it is difficult to provide an artificial flower design wherein a seemingly real aroma or fragrance is emitted from the flower.

Therefore, there is and continues to be a need for an artificial flower design that is compact, practical to manufacture, and one that is capable of emitting a fragrance or scent that approximates flowers.

### SUMMARY OF THE INVENTION

The present invention relates to an artificial flower, tree or wreath. In one embodiment, an artificial flower includes one or more stems with each stem including an artificial pedestal secured to one end thereof. An electric fan-fragrance unit includes an electric fan, a fragrance housing and fragrance source disposed in the housing. The fragrance housing is disposed adjacent the electric fan and includes an air inlet and an air outlet. The air inlet of the fragrance housing is positioned adjacent the fan such that upon actuation of the electric fan air is pulled into the air inlet of the fragrance housing, past the fragrance source and forms an air-fragrance mixture. The air-fragrance mixture moves out the air outlet of the fragrance housing into the electric fan which is operative to direct the air-fragrance mixture to one or more areas where the air-fragrance mixture is emitted from or about the artificial flower.

In one particular embodiment, there is provided a manifold and one or more hollow conduits. The air-fragrance mixture is directed from the electric fan to the manifold which is in turn connected to the one or more hollow conduits. The air-fragrance mixture is directed from the manifold into the series of hollow conduits which form a part of an artificial flower, tree or wreath or extend thereabouts. Ultimately the air-fragrance mixture carried by the hollow conduits is emitted about the artificial flower, tree or wreath.

The present invention also entails a fragrance delivery system for an artificial flower, tree or wreath. This artificial fragrance delivery system includes a support structure for supporting a fragrance source. An electric fan is provided that induces a system or stream of air past the fragrance source forming an air fragrance mixture. This air fragrance mixture is directed into one or more hollow conduits that form a part of an artificial flower, tree or wreath or which extends about or adjacent an artificial flower, tree or wreath. Ultimately the air-fragrance mixture carried by these hollow conduits is emitted about the artificial flower, tree or wreath.

### BRIEF DESCRIPTION OF THE DRAWINGS

FIG. 1 is a sectional view of the artificial flower of the present invention showing one embodiment of the present invention where a fragrance source is disposed in the hollow stem of the flower.

FIG. 1A is an enlarged fragmentary sectional view of a portion of the stem of the artificial flower having the fragrance source therein.

FIG. 2 is a sectional view similar to FIG. 1 but with a different fragrance source than that shown in FIG. 1.

FIG. 2A is an enlarged sectional view of a potion of the stem of the artificial flower shown in FIG. 1 having the fragrance source disposed therein.

FIG. 3 is a view similar to FIGS. 1 and 2, but illustrating another fragrance source for the artificial flower.

FIG. 3A is an enlarged sectional view of the portion of the stem of the artificial flower shown in FIG. 3 and which shows the fragrance source disposed therein.

FIG. 4 illustrates an artificial flower similar to the one shown in FIG. 1, but with a power supply externally connected to a bulb portion of the stem.

FIG. 4A is an enlarged sectional view of the stem of the artificial flower shown in FIG. 4 that shows the fragrance source disposed therein.

FIG. 5 is a sectional view of an alternate design for an artificial flower where a fragrance source is disposed in a detachably coupled flower portion.

FIG. 5A is a sectional view of the flower portion of FIG. 5.

FIG. 6 illustrates a partial cross-section of an artificial flower arrangement.

FIG. 7 illustrates artificial potpourri according to an exemplary embodiment of the present invention.

FIG. 8 illustrates a sectional view of another exemplary embodiment of artificial potpourri according to the present invention.

FIG. 8A is an enlarged sectional view of a potpourri flower of FIG. 8.

FIG. 9 illustrates artificial potpourri according to another exemplary embodiment of the present invention.

FIG. 9A illustrates an enlarged sectional view of a potpourri flower of FIG. 9.

FIG. 10A illustrates an artificial flower disposed within a car according to an exemplary embodiment of the present invention.

FIG. 10B illustrates another artificial flower disposed within a car according to an exemplary embodiment of the present invention.

FIG. 10C illustrates another artificial flower disposed within a car according to an exemplary embodiment of the present invention.

FIG. 11 is a perspective view of an alternate embodiment for an artificial flower.

FIG. 12 is an exploded view of a portion of the artificial flower shown in FIG. 11.

FIG. 13 is a fragmentary perspective view showing the electric fan mounted adjacent the manifold.

FIG. 14 is a view similar to FIG. 13 but showing a fragrance housing added to the assembly.

FIG. 15 is an exploded view showing the manifold, a portion of a stem, and a petal.

FIG. 16 is a bottom plan view of a petal of the artificial flower of the present invention.

FIG. 17 is a fragmentary perspective view showing a stem and a branch extending from the stem wherein the stem is secured to the manifold.

### DESCRIPTION OF EXEMPLARY EMBODIMENTS OF THE INVENTION

With further reference to the drawings, the artificial flower of the present invention is shown therein and indicated generally by the numeral 10. The artificial flower 10 includes a hollow stem indicated generally by the numeral 12 and a flower portion 16 secured to or extending from the upper portion of the stem 12. In the context of this application, the term “artificial” simply means non-living. Thus, the artificial flower 10 can be made of various materials such as plastics, metal, synthetic materials, or could comprise dried flowers or dried vegetation.

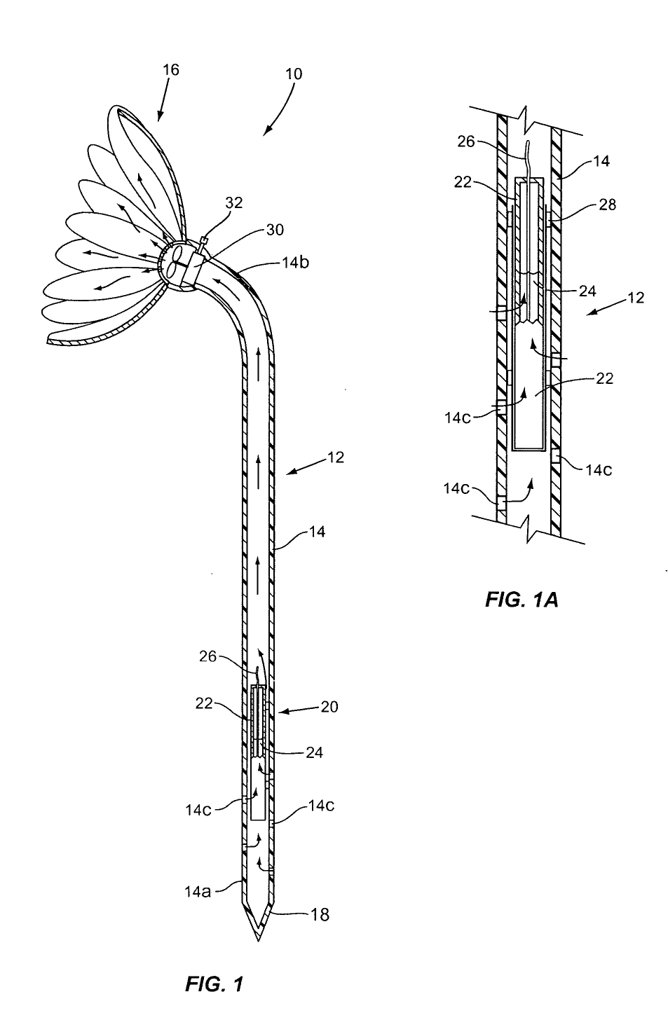

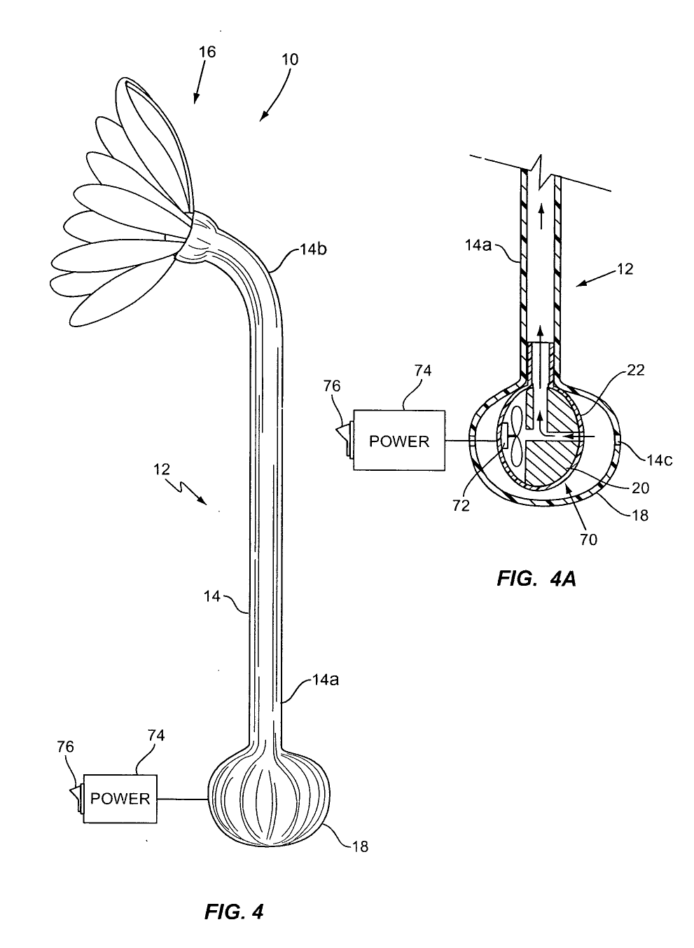

As seen in the drawings, the stem 12 is hollow. Stem 12 includes a surrounding wall structure 14 and a lower portion 14a that extends to an anchor end 18. As seen in FIGS. 1-3, anchor end 18 is formed into a point that permits the artificial flower 10 to be staked or spiked into a support material such as Styrofoam, potting soil, dirt or other supporting structure. Alternately, anchor end 18 may be formed into a bulb shape (see FIG. 4) similar to that of a real flower.

Opposite the lower portion 14a is an upper portion 14b. It is noted that the upper portion 14b of stem 12 is disposed adjacent the flower portion 16. The end of stem 12 about the upper portion 14b may be open or partially closed. In the embodiment illustrated in FIGS. 1-3, the end of the upper portion 14b of the stem 12 includes a rounded end that includes a series of openings formed therein. As will be described subsequently herein, the stem is designed such that air and a fragrance can move therethrough and, in at least one embodiment, is designed such that the fragrance can be emitted or dispersed from the upper portion 14b of the stem 12 into an environment where the flower portion 16 of the artificial flower 10 resides. Also, it is appreciated that the wall structure 14 of the stem 12 may include one or more openings 14c along the length of stem 12. Again, as will be appreciated from subsequent portions of this disclosure, openings 14c within the stem 12 may permit air to enter the stem 12 and move upwardly through the hollow stem 12 towards the flower portion 16.

Flower portion 16 is disposed adjacent the upper portion 14b of the stem 12. Again, the flower portion 16 forms a part of the artificial flower 10 and in the particular embodiments illustrated herein, the flower portion 16 extends from the upper portion of the stem. It is appreciated that flower portion 16 may be secured or integrally formed with the stem 12 through various manufacturing and fabrication techniques. Flower portion 16 may also assume various shapes and configurations. In some embodiments, it is contemplated that the flower portion 16, as illustrated in the drawings, would form a generally cup shape and comprise a series of petals.

The present invention entails associating a fragrance source, indicated generally by the numeral 20, with the artificial flower 10 for dispersing fragrance into the environment. The fragrance source 20 can be of various conventional types. Further, the particular scent emitted by the fragrance source 20 may vary and may be selected to simulate or mimic the smell or scent of various flowers. In addition, the size of the fragrance source 20 and/or the size of the air intake openings and/or outtake openings may affect the amount of fragrance dispersed into the environment.

Those skilled in the art will appreciate that the consumer may control any or all of these parameters and characteristics. For example, a consumer may select a particular scent by selecting one or more fragrance sources 20 for one or more artificial flowers 10. The consumer may also control the amount of dispersed fragrance by opening a slidable panel (not shown) to expose more openings in the container 22 or by varying the size of the intake openings. In addition, the amount or size of the fragrance source 20 can be varied. For example, in embodiments utilizing a solid fragrance source, the present invention may employ various sizes of such fragrance sources 20. In cases where a liquid or a semi-liquid fragrance is used, the quantity of the fragrance source 20 held within the artificial flower 10 can be varied.

In the embodiment illustrated in FIG. 1, fragrance source 20 comprises a container 22 having a selected liquid fragrance 24 contained therein. Container 22 is preferably sealed but includes an opening for receiving a wick 26. Wick 26 extends downwardly into container 22 and includes a portion that is submerged within the liquid fragrance 24. A portion of the wick 26 extends from the top of the container 22 and is exposed. Container 22 can be disposed in various locations about the artificial flower 10. In the embodiment illustrated in FIG. 1, container 22 is disposed within the hollow stem 12.

Various mounting structures or mounting techniques can be utilized. For example, the container 22 can be set or held in an open mounting structure 28 that is frictionally supported between the interior walls of the stem 12. In the case of the embodiment shown in FIG. 1, the open mounting structure 28 is of an open plastic frame that basically slides into the stem 12 and is frictionally held therein. It will be appreciated that the open mounting structure 28 may alternatively be secured to the walls 14 of the stem 12 by adhesive or by any other known securing means. It may also be beneficial in certain embodiments for the mounting structure 28 to be designed such that there is formed at least one air passageway between the container 22 and the interior walls of the stem 12. In other words, it will be beneficial in certain embodiments to provide an open space between the container 22 and the interior walls to allow air to pass upwardly around the container 22 and over the wick 26.

In the case of the embodiment shown in FIG. 1, there is provided a fan 30 or impeller disposed in the upper end portion 14b of the stem 12. The fan 30 is a battery-powered fan that includes a main body held and supported within the upper portion 14b of the stem and including a fan blade, impeller, or propeller extending therefrom. A switch 32 may extend from the main body of the fan 30 outwardly through a sidewall of the stem 12. While not shown, those skilled in the art will appreciate that the power source 74 for the fan 30 may be disposed internally or externally to the artificial flower 10. Further, as shown in FIG. 6 and discussed further below, anchor end 18 may electrically connect to an external power source when staked or held within support material. In addition, alternate embodiments of the present invention may also include power adapters so that fan 30 may plug into a wall outlet (not shown) or a vehicle power port.

According to the present invention, fan 30 may provide a fixed airflow rate. Alternatively, fan 30 may provide a variable airflow rate. For example, fan 30 may be a multi-speed fan that enables a consumer to select the amount of fragrance dispersed into the environment by selecting a fan speed. In a preferred embodiment, a consumer selects a fan speed by positioning switch 32 in the desired position.

In the case of the design shown in FIG. 1, the fan 30 is disposed above the fragrance source 20. Therefore, the impeller or fan blade associated with the fan 30 is designed to induce or pull air from below the container 22, past the container 22 and over the wick of 26. The fan causes air to be induced through the openings 14c into the interior of the stem 12. Once in the stem 12, the induced air is pulled upwardly past the container 22 and the wick 26. Accordingly, fragrance on the saturated or wet wick 26 will be transferred to the passing air and ultimately will be dispersed out the upper portion 14b of the stem 12 adjacent the flower portion 16. Note also that the main body or frame of the fan 30 would be provided with openings that would enable air to be moved or pulled through the upper portion 14b of the stem 12, through the fan structure, and out of the upper end of the stem 12 to where the fragrance-laden air disperses into an area occupied in part at least by the flower portion 16.

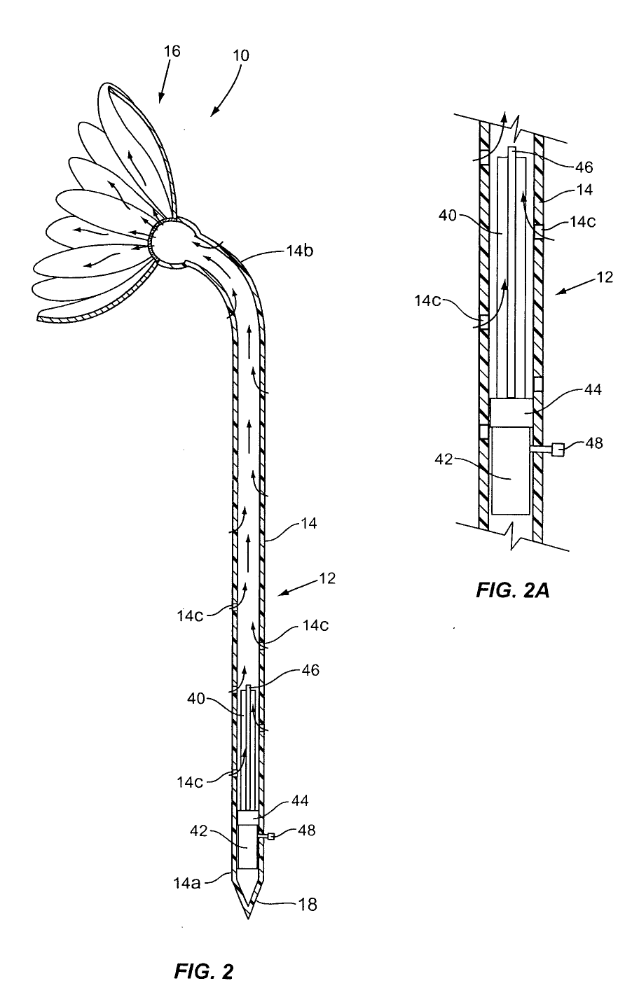

Turning to FIG. 2, an alternative embodiment for the artificial flower 10 is shown therein. In this case, the fragrance source 20 is in the form of a fragrance gel or block 40. It will be appreciated that fragrance blocks are known in the air freshener art and therefore, details of such will not be submitted herein because those skilled in the art will understand the basic structure and composition of conventional fragrance blocks. For example, see U.S. Pat. No. 6,289,176, the disclosure of which is expressly incorporated herein by reference. In any event, fragrance block 40 is disposed within the stem 12 of the artificial flower, as shown in the embodiment of FIG. 2. The fragrance block 40 may assume different configurations. In the case of the embodiment illustrated herein, fragrance block 40 is elongated and round and is in the form of a generally cylindrical shape. Further, fragrance block 40 includes a central opening.

The artificial flower 10 may also include a heater 42, where fragrance block 40 is supported in the stem 12 over the heater 42. Heater 42 may be battery-powered and may include a switch 48 that extends from the heater 42 out the sidewall of the stem 12. As with the fans described above, heater 42 includes a power source (not shown) that may reside within or externally from the artificial flower 10 and may include a power adapter (not shown) to allow heater 42 to plug into a wall outlet. Further, as with the fans 30 described above, heater 42 may provide a fixed amount of heat or may provide a variable amount of heat based on the position of switch 48. A mounting block 44 disposed over the heater and a heating element 46, such as resistive heating element, extends upwardly from the heater 42 and the mounting block 44 and extends through the central opening formed in the fragrance block 40.

Further, the side wall structure 14 of the stem 12, especially in the area adjacent the position of the fragrance block 40, will includes a series of openings 14c therein to allow air to be induced or to naturally flow into the stem 12. That is, the fragrance block 40 would be preferably spaced inwardly from the wall structure 14 of the stem 12 so as to allow air to pass between the fragrance block 40 and the interior walls of the stem 12. Additionally, a fan 30, such as shown in FIG. 1, can be positioned below or above the fragrance block 40 to induce air into the stem 12 and over the fragrance block 40. When the heater 42 is turned on, heating element 46 heats the fragrance block 40 and cause the fragrance block 40 to vaporize and produce a vaporized scent or aroma.

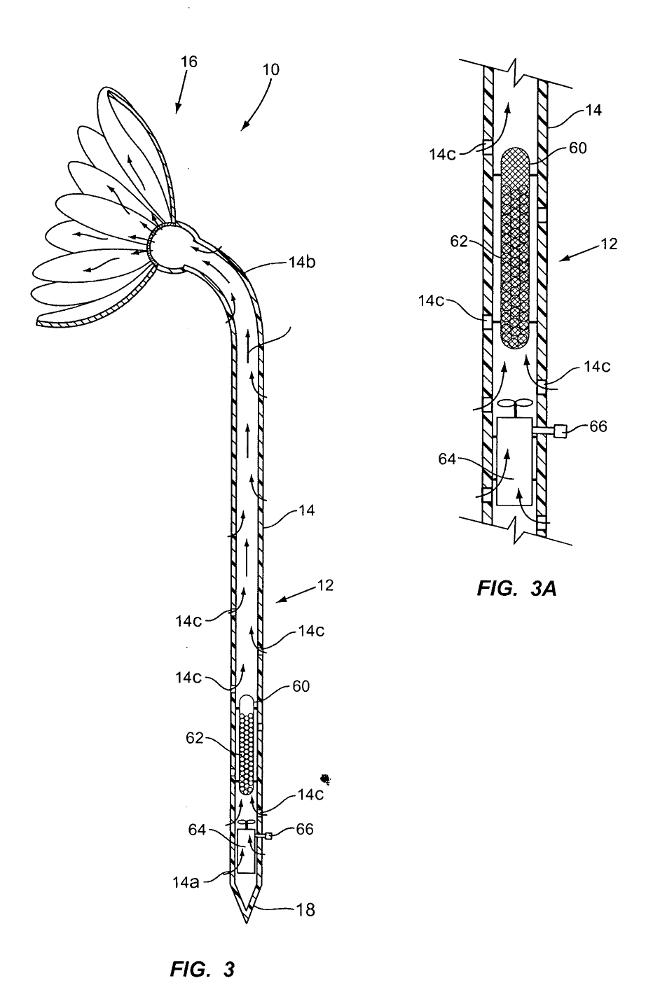

Turning now to FIG. 3, another embodiment of the present invention is shown therein. In this case, container 22 is a permeable container 60. Permeable container 60 may assume various forms. For example, permeable container 60 may comprise a plastic container with openings formed therein that enable air to circulate through the permeable container 60. Disposed within the permeable container 60 is a fragrance source 20, such as a fragrance gel or an array of fragrance pellets 62. Each fragrance pellet 62 may comprise a fragrance particle or ball and would over time emit a desired fragrance. Preferably the permeable container 60, including the fragrance pellets 62, would be supported within a mounting structure secured in the stem 12. In one embodiment, the mounting structure is similar to that discussed above with respect to the mounting structure utilized to hold and support the container 22. In any event, the function of the mounting structure would be able to hold the permeable container 60 within the stem 12. Preferably the mounting structure would be of an open frame design that would, when inserted within the stem 12, provide an opening around the permeable container 60 to allow air to flow past. In some embodiments, it is foreseen that the permeable container 60 would be frictionally retained or held within the stem 12 and would effectively assume substantially the entire cross section of the stem about a selected length of the stem. In this case, air moving from below the permeable container 60 upwardly would be forced to pass through the permeable container 60.

In any event, the design of FIG. 3 may also include a fan 64 with a propeller, impeller, or blade associated therewith. Fan 64 in this embodiment is disposed below the permeable container 60, however, it is understood that the fan 64 could be oriented above the permeable container 60, for example, as indicated in FIG. 1. Fan 64 may include a switch 66 that extends outwardly therefrom through the wall structure 14 of the stem 12. Again, as was the case with the fan shown in FIG. 1, fan 64 may be battery-powered and the main body of the fan 64 would generally be of an open construction that allows air to be pulled from below the fan 64, through the open main body of the fan 64, and upwardly through the stem 12.

Still another embodiment of an artificial flower of the present invention is illustrated in FIG. 4. As with the embodiments described above, artificial flower 10 includes the hollow stem 12 and the flower portion 16 secured to or extending from the upper end of the hollow stem 12. In this embodiment, however, anchor end 18 of stem 12 forms the shape of a bulb. Therefore, it can be said that the artificial flower 10 includes a lower bulb portion 18.

Bulb portion 18 secures to the stem 12 at the base of the lower portion 14a of stem 12 by any means known in the art, including friction and/or by the use of adhesives and/or fastening devices. Further it is contemplated that the bulb portion 18 could be integrally formed with the stem 14. The bulb portion 18 forms an internal cavity, indicated generally by the numeral 70. Disposed within the cavity 70 is a housing structure 22 that includes a diffuser or fan 72 and a fragrance source 20. While the illustrated embodiment illustrates a fan 72, those skilled in the art will appreciate that a heater may be used in place of or in addition to the fan 72.

Formed in the housing 22 is an air passage that, as seen in FIG. 4A, allows air to enter the housing 22 and pass through and into contact with the fragrant source 20 after which the air is directed through the hollow stem 14. It is noted in FIG. 4A that the bulb portion 18 includes at least one air inlet 14C for permitting air to enter the internal cavity 70. Fan 72 electrically connects with a power source 74 that includes an on/off switch 76. In a preferred embodiment, the power source 74 is a battery power source. However, power source 74 may include a power adaptor that can be plugged into an electrical outlet.

When switched on, the power source activates the fan 72 such that air circulates through and around fragrance source 20. As described above, fan 72 may provide a fixed rate air flow or a variable rate air flow. The scented air then travels through the hollow stem 12 and exits the artificial flower 10 at the flower portion 16. While the embodiment illustrated in FIGS. 4 and 4A shows a container 22 that includes both the fan 72 and the fragrance 20, those skilled in the art will appreciate that the present invention is not so limited. For example, fan 72 may be housed separately from the container 22 within anchor end 18.

In the embodiment illustrated in FIGS. 4 and 4A, fragrance source 20 comprises a fragrance block or gel. However, the present invention is not so limited. Container 22 may contain any known fragrance source 20, including the fragrance sources described above. For example, container 22 may contain a liquid fragrance 24, where a wick 26 positioned in the liquid fragrance 24 extends from the fragrance container 72 (similar to FIG. 1). Alternately, container 22 may comprise a permeable container 60 with fragrance gel or pellets 62 disposed therein (similar to FIG. 3).

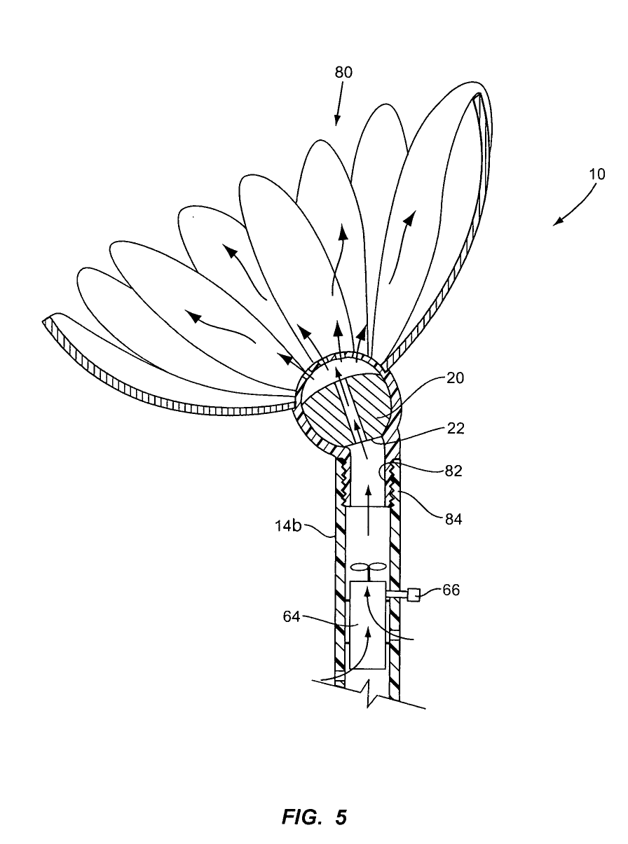

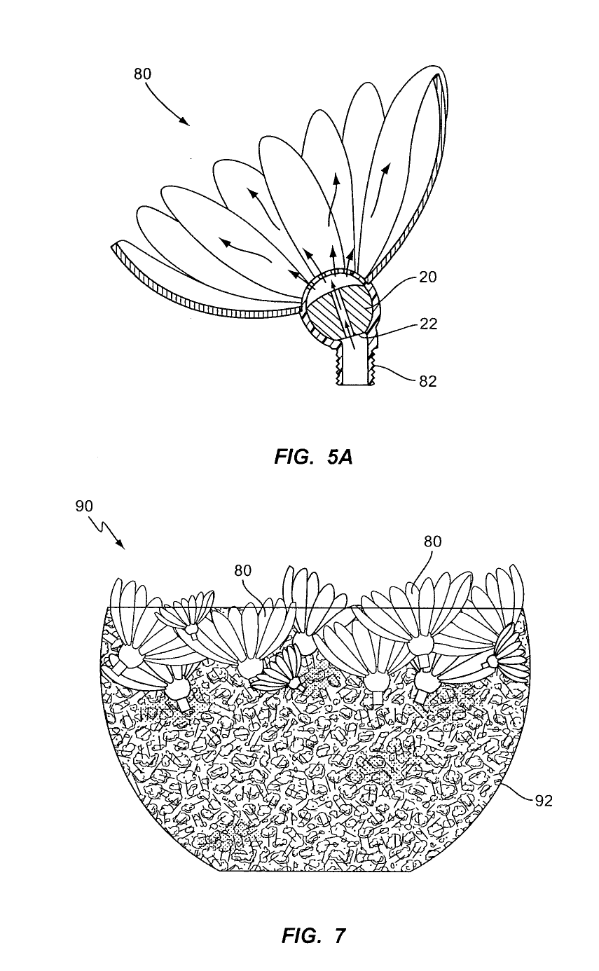

Referring now to FIG. 5, another embodiment of the invention will be described herein. In the illustrated embodiment, artificial flower 10 comprises a flower portion 80 that detachably couples to the hollow stem 12. Flower portion 80 also includes a fragrance source 20 that may be disposed in a container 22 secured within flower portion 80 by any of the means described above. As a result, the fragrance provided by fragrance source 20 may be dispersed to the surrounding environment through evaporation and natural airflow.

Alternatively, a diffusion source, such as a fan 64 and/or a heater (not shown), may be positioned within the artificial flower to disperse the scented air. For example, fan 64 may be disposed within the upper end 14b of the hollow stem 12 proximate the flower portion 80. As described above, the fan induces airflow around and/or through the fragrance source, causing scented air to be emitted from the flower portion 80.

As seen in FIG. 5A, the detachable flower portion 80 includes a base that is generally disposed about the bottom of the flower portion 80. As will be discussed below, the base portion lies just above a fastener or connector that is adapted to attach the flower portion 80 to the stem 12 of the artificial flower 10. In any event, as seen in FIG. 5A, the base portion of detachable flower portion 80 includes a hollow cavity for receiving and holding a container or housing 22 that contains or holds the fragrance source 20. Note in FIG. 5A where at least one air passageway extends through the container 22 and through the fragrance source 20. Note also that the upper portion of the base, above the fragrance source 20, includes a series of openings for dispersing the air among the petals of the detachable flower portion 80.

As mentioned above, flower portion 80 detachably couples to stem 12. Typically, a connector 82 disposed on a bottom end of the flower portion 80 detachably couples to a corresponding connector 84 disposed on the upper end 14b of stem 12. In an exemplary embodiment, connector 82 comprises a threaded section at the base of flower-portion 80 that threadably connects to a corresponding threaded section of a connector 84 at the top of the upper end 14b of stem 12. Of course, alternate connectors, such as snaps, straps, etc., may be used. Further, flower portion 80 may simply secure to stem 12 via friction.

Because flower portion 80 detachably couples to the stem 12, a consumer may replace or change the fragrance source 20 at any time simply by removing the former flower portion 80 and attaching a new flower portion 80, which includes a new fragrance source 20, to the stem 12. Alternatively, the consumer may refill container 22 with a new fragrance source 20. In still another embodiment, the consumer may remove the container 22 from the flower portion 80 and couple a new container 22 within flower portion 80. In any event, the artificial flower 10 of the present invention allows the consumer to exchange and/or replenish the fragrance source 20 of artificial flower 80 without replacing the entire flower 10.

The artificial flowers 10 of the present invention may be used individually or may be arranged as an artificial flower bouquet 90 within a container 92, such as a vase or bowl, as shown in FIG. 6. FIG. 6 shows a plurality of artificial flowers 10 secured within a support structure 88 disposed within the container 92. The support structure 88 includes one or more air intakes 14c, a diffuser, such as a fan 64, and a power source 74 that electrically connects to the diffuser and optionally includes a switch 76. In one embodiment, each artificial flower 10 includes a fragrance source 20, as shown in any one of FIGS. 1-3. The artificial flowers then emit a fragrance when the fan 64 pushes air through the hollow stems 14 and across the fragrance sources 20 disposed within the artificial flowers.

In an alternate embodiment, the fragrance source 20 may be disposed within support structure 88 proximate the air intake 14c. In this embodiment, the air is scented by fragrance source 20 before entering the fan 64. Fan 64 provides enough air flow to push the scented air through the hollow stems 14 of the artificial flowers 10 such that scented air is emitted from the bouquet of artificial flowers 10.

FIG. 7 illustrates yet another embodiment of the present invention where a collection of the flower portions 80 shown in FIG. 5A may be used as potpourri 90. While FIG. 5A shows a flower portion 80 that includes the connector 82, it will be appreciated that some embodiments of the present invention may exclude the connector 82 when the flower portions 80 are used for potpourri.

In any event, potpourri 90 may be placed in any suitable container 92, as shown in FIG. 7, or may be scattered loosely on any surface. Potpourri 90 may also include additional artificial flowers or material that operates as filler and does not contribute to the fragrance being emitted from the potpourri 90. When collected as potpourri, each flower portion 80 contributes to the overall scent emitting from the potpourri 90. A consumer may therefore create any desired fragrance by either using flower portions 80 with the same desired fragrance or by combining two or more different flower portions 80 with different fragrances.

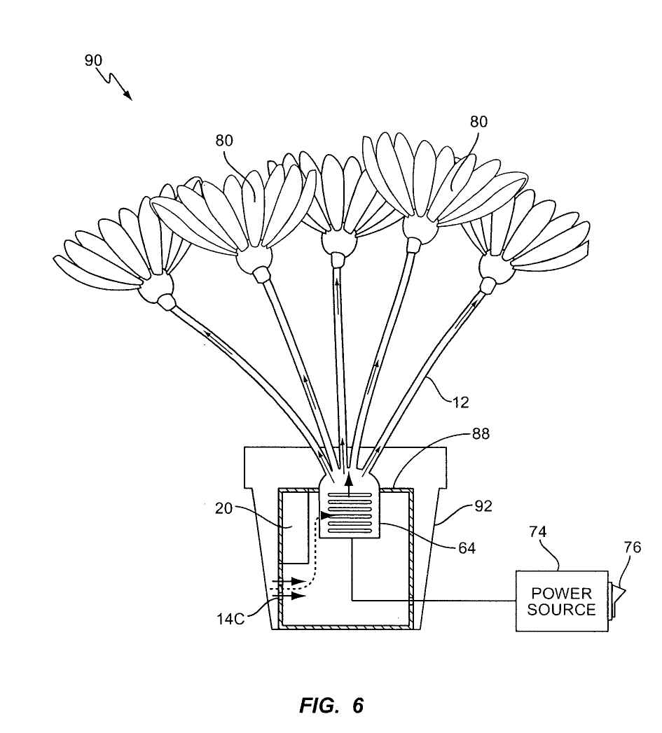

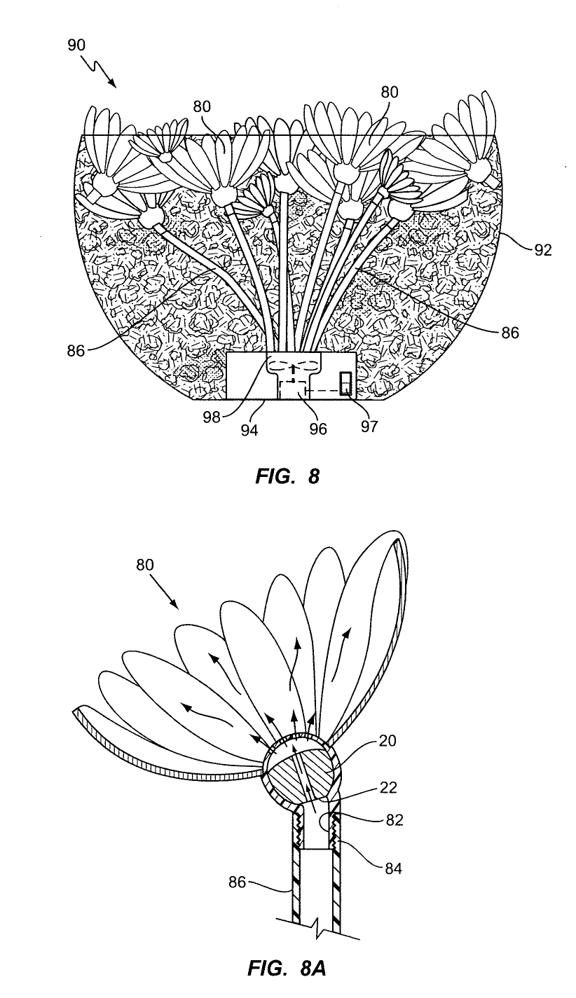

In exemplary embodiments, a scent diffuser 94 may be disposed in a container 92 as shown in FIG. 8. Scent diffuser 94 includes a fan 96 (or any other known diffuser as discussed above) electrically connected to a switch 97. Hollow tubes 86, made of a flexible or rigid material, couple to one or more openings 98 in diffuser 94 at a first end and couple to one or more flower portions 80 at an opposite end. Hollow tubes 86 may be detachably or fixedly coupled to the openings 98 according to any known method. Further, as shown in FIG. 8A, one or more flower portions 80 may detachably couple to the hollow tubes 86. As a result, airflow generated by fan 96 exits the openings 98 in diffuser 94, flows through the hollow tubes 86, circulates around and/or through the fragrance sources 20 disposed in the flower portions 80, and causes scented air to disperse from the potpourri 90.

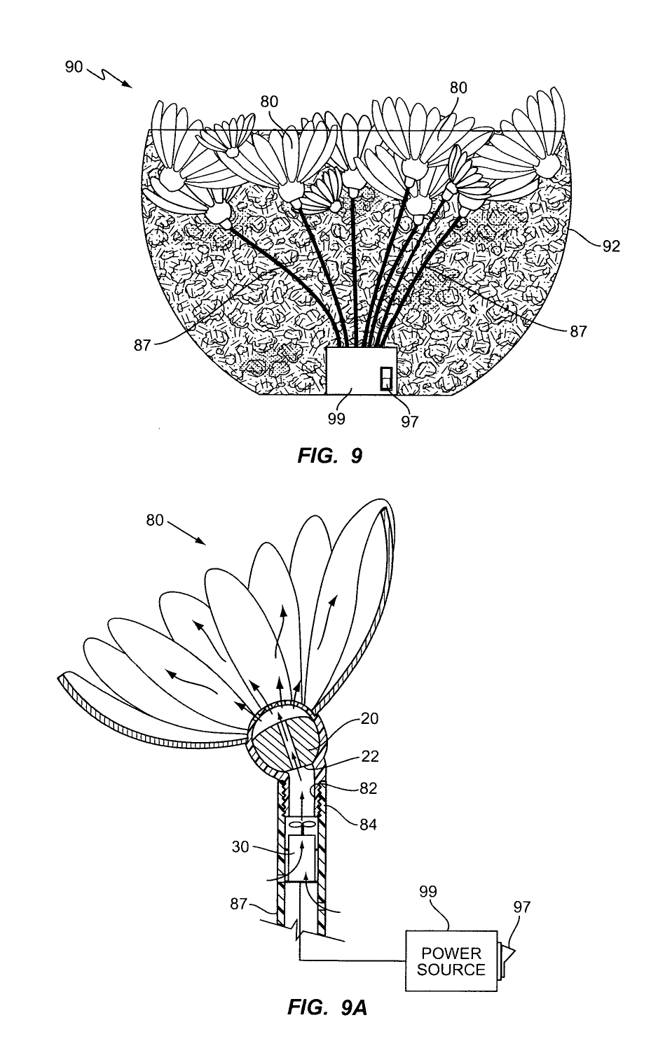

Alternatively, as shown in FIGS. 9 and 9A, each flower portion 80 may include the fragrance and a diffuser, such as a fan 30. In this embodiment, electrical wires 87 detachably connect the flower portions 80 to a power source 99 disposed within the container 92. As discussed above, one or more batteries or a power adapter may make up the power source 99. When the power source 99 is activated, the electrical wires 87 carry the necessary electrical current to power the fans 30 disposed within the flower portions 80, causing air to flow over the fragrance sources 20 and scented air to disperse from the potpourri 90.

Those skilled in the art will appreciate that the flower portions 80 in the potpourri that detachably couple to the hollow tubes 86 or to the electrical wires 87 may be replaced by removing the former flower portion 80 and attaching a new flower portion 80. As a result, a consumer may replace former flower portions 80 with new flower portions 80 any time the consumer wishes to refresh the potpourri scent or to replace the potpourri scent with a new scent.

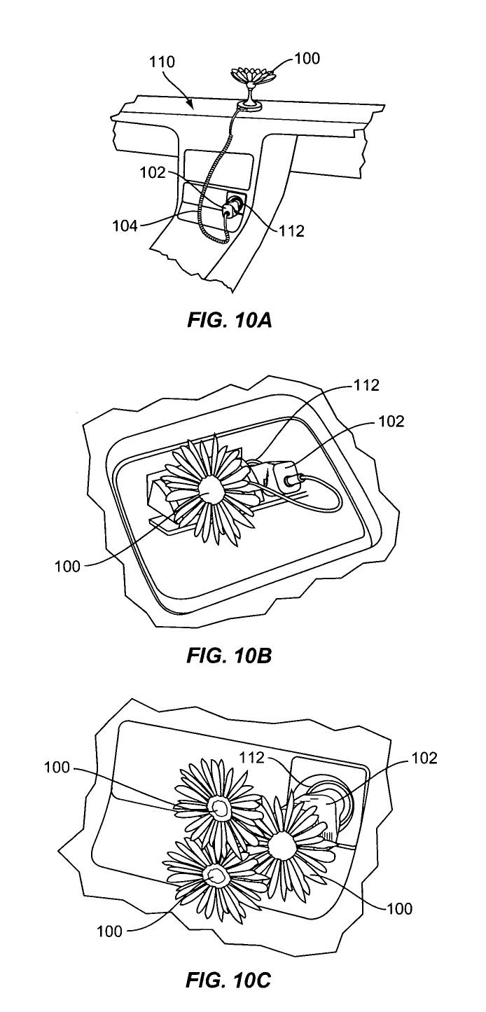

FIGS. 10A-10C illustrate still another embodiment of the present invention. As with the embodiments described above, the artificial flower 100 of FIGS. 10A-10C include a fragrance source and a diffuser, such as a fan or a heater, disposed within the artificial flower 100. However, unlike the embodiments described above, a car battery provides the power for the diffuser via an adapter 102. One end of the adapter 102 detachably couples to the diffuser, while the other end detachably couples to a power socket 112 within a car, such as a cigarette lighter socket, via a power cord 104.

As shown in FIGS. 10A-10B, the artificial flower 100 may include a mounting device or structure for mounting the artificial flower 100 to the dashboard 110 or to an adjacent ashtray. For example, the mount structure may include a suction cup or other semi-permanent mounting device that would function to secure the base of one or more artificial flowers 100 to the dashboard 110, to an ashtray, or to other surfaces found in a vehicle.

FIG. 10C shows another alternate design wherein one or more artificial flowers 100 are integrated together and supported directly or indirectly from an adapter 102 that fits into the cigarette lighter socket 112. Various support structures or devices can be utilized to support the artificial flowers 100 in various configurations.

It is appreciated that the present invention presents an artificial flower that is designed to emit a pleasing fragrance or aroma. A fragrance source associated with the flower is designed to emit a fragrance that will be dispersed about the flower. Although the fragrance source can be disposed in various positions and locations with respect to the artificial flower, in some embodiments, the fragrance source is disposed in the hollow stem that forms a part of the artificial flower. In other embodiments, the fragrance source is disposed in the flower portion of the artificial flower. Moreover, the fragrance source may simply be designed or selected to be of the type that will slowly and over a period of time simply emit a fragrance that will move through or from the artificial flower. Dispersion of the fragrance or scent can be enhanced by utilizing a heater to heat the fragrance source and/or a fan to pull or push air past the fragrance source.

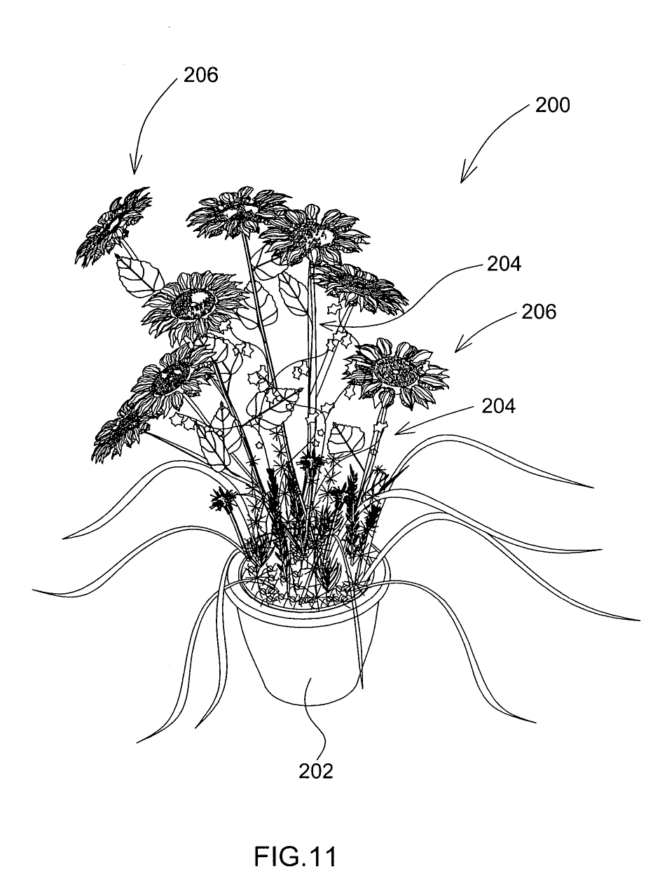

Now turning to FIG. 11, there is shown there an alternate design or embodiment for an artificial flower. This alternate artificial flower design is indicated generally by the numeral 200. Artificial flower 200 includes a flowerpot 202. Supported in the flowerpot is an artificial flower arrangement that comprises a series of stems with each stem being indicated generally by the numeral 204. Secured or disposed about a top portion of respective stems 204 is a series of petals indicated generally by the numeral 206. To give the artificial flower 200 a real life appearance, accompanying vegetation can also be provided, especially around the base of the flower arrangement and about the top of the pot 202. As used herein, the term artificial flower means any artificial flower, plant, tree, wreath, or shrub.

Artificial flower 10 includes a fragrance or scent delivery system. As will be appreciated from subsequent portions of this disclosure, the fragrance or scent delivery system is designed to generate or form an air-fragrance mixture and to direct or channel that air-fragrance mixture to or through portions of the artificial flower where the air-fragrance mixture is ultimately emitted from the artificial flower or emitted from an area around or in the vicinity of the artificial flower. The fragrance or scent selected emulates or resembles fragrances or scents emitted by living flowers, plants, trees, etc.

Artificial flower 200 is provided with a fragrance support structure. The fragrance support structure supports or holds a fragrance source 214. Fragrance source 214 can conform to various types of fragrances. In one embodiment, fragrance source 214 can comprise a series of small fragrance beads. As air is passed over and around the beads, the fragrance of the beads is mixed with the air to form an air-fragrance mixture. In the embodiment illustrated herein, the fragrance support structure comprises a fragrance housing 208. Fragrance housing 208 includes a small plastic or metal container having an interior area that receives and holds the fragrance source 214. As illustrated in the drawings, fragrance housing 208 includes a perforated air inlet 210 and a perforated air outlet 212. Accordingly, air can pass into the fragrance housing 208 via the inlet 210 and the air-fragrance mixture formed or made up in the fragrance housing can be exhausted from the housing via the outlet 212.

As will be appreciated from subsequent portions of the disclosure, in the design illustrated herein, fragrance housing 208 is designed to be connected or disposed adjacent an electric fan that is indicated generally by the numeral 220. To accomplish this, fragrance housing 208 includes a pair of connectors 216 that project therefrom. In this case, connectors 216 include a pair of stud connectors that are designed to be inserted into openings formed in the housing structure of the electric fan 220.

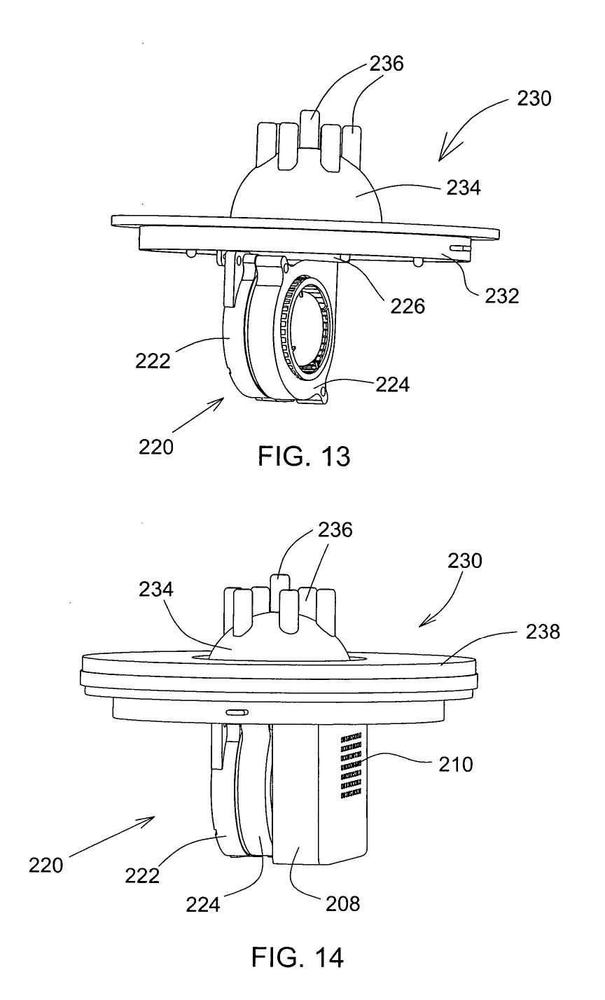

Electric fan 220 includes a housing structure. The housing structure of the electric fan 220 includes a pair of housings 222 and 224 that are secured together. Forming a part of the electric fan 220 is a motor and fan unit 228. Motor and fan unit 228 is housed within the housing structure and is adapted to be electrically driven. In the case of the electric fan disclosed herein, the same comprises a small squirrel cage-type electric fan that is well known in the art. Note in FIG. 13 where the electric fan includes an air inlet formed on the right side thereof, as viewed in FIG. 13.

Disposed over electric fan 220 is a manifold assembly indicated generally by the numeral 230. Manifold assembly 230 includes a base 232 that can be constructed of plastic or other suitable material. Forming a part of the base 232 is a chamber 234 that can be constructed of plastic, rubber or other suitable materials. Extending from chamber 234 is a series of outlets 236. Outlets 236 are sometimes referred to as stub outlets. Disposed over the base 232 and around the chamber 234 is a collar 238 that, again, can be constructed of plastic, rubber or other suitable materials. Collar 238 includes an upper surface that can be made to simulate vegetation, grass or the like.

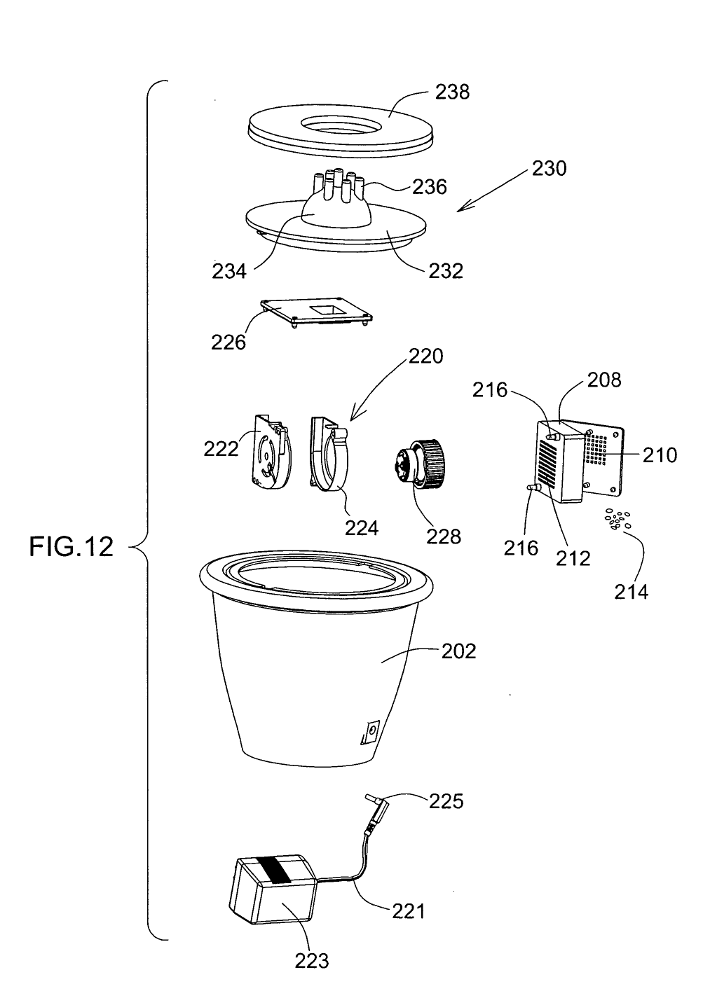

Disposed below the base 232 is a plate or interface 226 (see FIG. 12). Plate 226 includes an opening formed therein. The upper surface of plate 226 is glued or otherwise secured to the bottom of base 232. The opening formed in plate 226 is aligned with a chamber 234. In addition, the opening formed in the plate 226 is aligned with an opening formed in the top of the housing of the electric fan 220. The opening in the top of the electric fan 220 is sealed against the bottom surface of the plate 226. The upper surface of the plate 226 is sealed against the bottom of the base 232. Thus, there is generally a sealed, airtight relationship between the plate 226 and the bottom of base 232, as well as a sealed relationship between the lower surface of the plate 226 and the opening formed in the top of the fan 220. Consequently, air being induced into the fan 220 is constrained or caused to move upwardly through the opening in the plate 226 into the chamber 232.

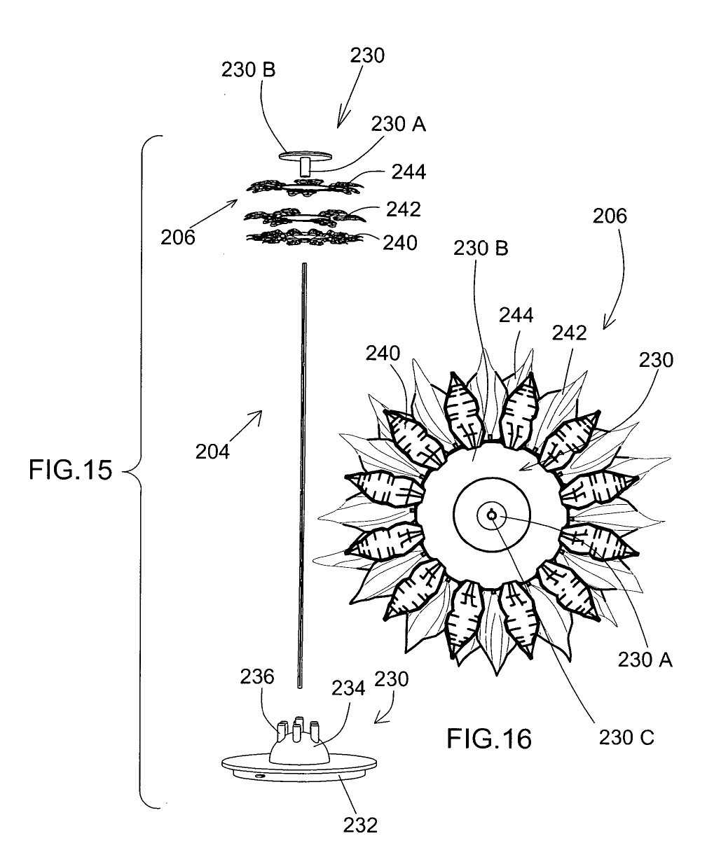

Stems 204 discussed above form hollow conduits for channeling or directing the air-fragrance mixture from the manifold 230. In particular, the respective stems 204 are connected in a sealed relationship to the stub outlets 236 extending upwardly from the chamber 234. The respective stems 234 extend upwardly from the manifold 230 and include upper end portions. Petals 206, discussed above, are secured to the upper end portions of the respective stems. Various petal designs can be used. In this case each petal includes a series of petal layers 240, 242 and 244. These petal layers are overlaid, as illustrated in FIG. 15, to produce the petals 206 shown in FIG. 11. Viewing each petal 206 in more detail, in the case of this design, each petal includes a disperser 230 that is designed to disperse the air-fragrance mixture. Viewing disperser 230, the same includes a stem 230A, a main body 230B and an outlet 230C. Dispenser 230, and particularly stem 230A, is designed to be secured to the upper portion of respective stems 204. The air-fragrance mixture passing through the stems 204 is directed through the dispenser 230 and out the outlet 20C. Thus, the fragrance or scent generated by the fragrance source 214 is emitted, in this case, from the petals 206.

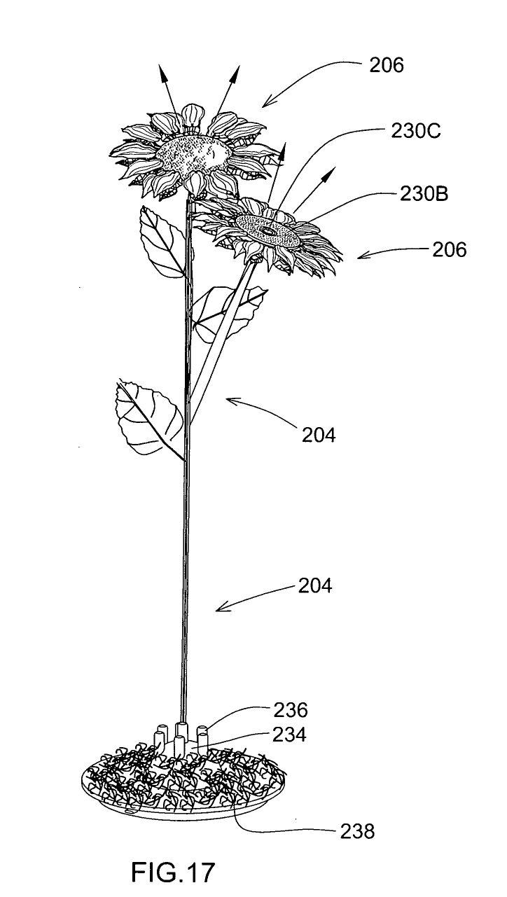

In the embodiment illustrated in FIGS. 11-17, the scent or fragrance delivery system has been described in conjunction with an artificial flower. However, it is understood that the same fragrance or scent delivery system can be incorporated into an artificial tree such as a Christmas tree or a wreath, or other types of artificial plants, shrubs or vegetation. As used herein, the term artificial flower is meant to encompass an artificial tree such as a Christmas tree or an artificial wreath such as a Christmas wreath.

From the foregoing discussion, it is appreciated that the present invention provides an economical, practical and efficient artificial flower that includes an effective system for delivering and dispersing fragrance about the artificial flower. The present invention may, of course, be carried out in other specific ways than those herein set forth without departing from the scope and the essential characteristics of the invention. The present embodiments are therefore to be construed in all aspects as illustrative and not restrictive and all changes coming within the meaning and equivalency range of the appended claims are intended to be embraced therein.

1. An artificial flower comprising:
a. one or more stems;
b. one or more artificial petals secured to the one or more stems;
c. an electric fan-fragrance unit including an electric fan, a fragrance housing, and a fragrance source disposed in the housing; and
d. wherein the fragrance housing is disposed adjacent the electric fan and includes an air inlet and an air outlet and wherein the air inlet of the fragrance housing is positioned adjacent the fan such that upon actuation of the electric fan air is induced into the air inlet of the fragrance housing, past the fragrance source to form an air-fragrance mixture that is exhausted out the air outlet of the fragrance housing into the electric fan:

2. The artificial flower of claim 1 wherein the fragrance housing is connected to the electric fan.

3. The artificial flower of claim 2 wherein the electric fan includes a housing and wherein the fragrance housing is secured to the housing of the electric fan.

4. The artificial flower of claim 1 wherein the electric fan includes an air inlet that is disposed directly adjacent the air outlet of the fragrance housing.

5. The artificial flower of claim 4 wherein the electric fan is a squirrel cage fan having a housing including at least one generally flat side and wherein the flat side includes an air inlet that is disposed directly adjacent the air outlet of the fragrance housing.

6. The artificial flower of claim 1 including a manifold having a series of outlets connected to the stems and wherein each stem includes a hollow portion; and wherein the electric fan is disposed with respect to the manifold such that the electric fan induces air through the fragrance housing and directs the air into the manifold which disperses the air into a series of stems.

7. The artificial flower of claim 6 wherein respective petals include an outlet that is communicatively connected to a stem such that air directed through the stems is dispersed through the outlet associated with the petals.

8. The artificial flower of claim 1 wherein the electric fan includes a housing and wherein the fragrance housing is connected to the housing of the electric fan by one or more studs that extend between the housing of the electric fan and the fragrance housing.

9. The artificial flower of claim 1 including a manifold having a chamber and a series of hollow stub conduits extending from the chamber and wherein the hollow stub conduits are connected to a series of stems where the stems have hollow portions for channeling air therethrough.

10. The artificial flower of claim 9 including a base for supporting the chamber of the manifold.

11. The artificial flower of claim 10 wherein the electric fan is disposed below the manifold and operative to direct air from the fragrance housing into the manifold where the air is dispersed through the hollow stems connected to the stub conduits of the manifold.

12. The artificial flower of claim 11 wherein the electric fan includes a housing having a top portion and wherein the top portion of the housing includes an outlet; and wherein the electric fan is disposed below the manifold and operative to direct air therefrom into the chamber of the manifold.

13. A method of dispersing fragrance from an artificial flower comprising:
a. inducing air into a fragrance housing and past a fragrance source in the fragrance housing to form an air-fragrance mixture;
b. directing the air-fragrance mixture into a manifold having a plurality of outlets;
c. directing the air-fragrance mixture into a plurality of hollow stems connected to the outlets of the manifold; and
d. directing the air-fragrance mixture from the stems to petals associated with the hollow stems such that the air-fragrance mixture is dispersed adjacent the petals.

14. The method of claim 13 including disposing an electric fan between the fragrance housing and the manifold such that the electric fan is operative to induce air into the fragrance housing and to direct the air-fragrance mixture therefrom to the manifold.

15. The method of claim 14 including connecting the fragrance housing to the electric fan such that the electric fan and fragrance housing are disposed in side-by-side relationship.

16. The method of claim 13 wherein the manifold includes a bladder and wherein the plurality of outlets of the bladder include a series of stub outlets that project from the bladder; and wherein an electric fan is disposed between the fragrance housing and the manifold with the electric fan including a housing having a top portion that lies generally below the manifold.

17. The method of claim 13 including an electric fan disposed between the fragrance housing and the manifold and wherein the electric fan includes a housing having at least one generally flat side having an air inlet formed therein; and wherein the fragrance housing includes a generally flat side having an air outlet formed therein and wherein the two flat sides of the electric fan housing and the fragrance housing are disposed in side-by-side relationship such that the air-fragrance mixture exiting the fragrance housing directly enters the air inlet of the electric fan.

18. The method of claim 13 including an electric fan disposed between the fragrance housing and the manifold and wherein the manifold, electric fan and fragrance housing forms a unitary structure and is supported within a receptacle.

19. A method of fabricating an artificial flower comprising: disposing a fragrance housing directly adjacent an electric fan wherein the fragrance housing includes an air inlet and an air outlet and wherein the electric fan includes an air inlet, and wherein the air outlet of the fragrance housing is disposed adjacent the air inlet of the electric fan such that the electric fan is operative to induce air into and through the fragrance housing and into the electric fan; connecting, directly or indirectly, an outlet of the electric fan with the manifold having a series of outlets associated therewith; and connecting the series of outlets of the manifold with a plurality of hollow stems such that an air-fragrance mixture leaving the fragrance housing is directed through the electric fan and into the manifold and into the hollow stems.

20. The method of claim 19 including connecting petals to the hollow stems.

21. A fragrance distribution system for an artificial flower comprising:
a. a fragrance support structure;
b. a fragrance source held by the fragrance support structure for emitting a fragrance;
c. an electric fan for inducing air past the fragrance source held by the fragrance support structure to form an air-fragrance mixture;
d. one or more hollow conduits forming a part of the artificial flower; and
e. wherein the electric fan directs the air-fragrance mixture into the hollow conduits where the air-fragrance mixture is routed about the artificial flower to where the air-fragrance mixture is dispersed.

22. The fragrance delivery system for an artificial flower of claim 21 where the fragrance support structure includes a housing that houses the fragrant source and includes an air inlet and an air outlet.

23. The fragrance delivery system for an artificial flower of claim 22 wherein the electric fan and fragrance housing are structurally attached.

24. The fragrance delivery system for an artificial flower of claim 23 wherein the electric fan is a squirrel cage fan having an air inlet that is disposed adjacent the air outlet of the fragrance housing.

25. The fragrance delivery system for an artificial flower of claim 22 including a manifold disposed between the electric fan and the hollow conduits and wherein the air-fragrance mixture is directed into the manifold by the electric fan and from the manifold the air-fragrance mixture is dispersed to the hollow conduits.

26. The fragrance delivery system for an artificial flower of claim 25 wherein the manifold includes a chamber and a series of stub outlets extending therefrom and wherein the respective hollow conduits are secured to the stub outlets such that the air-fragrance mixture directed to the manifold is directed from the manifold through the stub outlets and into the hollow conduits.

27. The fragrance delivery system for an artificial flower of claim 22 wherein the hollow conduits are incorporated into the artificial flower.

28. A method of delivering an air-fragrance mixture to an artificial flower comprising: supporting a fragrance source; utilizing an electric fan for inducing air to move past the fragrance source to form an air-fragrance mixture; directing the air-fragrance mixture into one or more hollow conduits that form a part of the artificial flower or extend adjacent the artificial flower; and directing the air-fragrance mixture through the hollow conduits so as to emit the air-fragrance mixture adjacent the artificial flower.

29. The method of claim 28 wherein the fragrance source is housed within a fragrance housing having an air inlet and an air outlet and wherein the method entails inducing air through the air inlet of the fragrance housing, past the fragrance source disposed therein and out the air outlet.

30. The method of claim 29 including directing the air-fragrance mixture from the air outlet of the fragrance housing directly into an inlet of the electric fan.

31. The method of claim 30 wherein the electric fan is a squirrel cage-type electric fan and includes an air inlet that is disposed directly adjacent the air outlet of the fragrance housing.

32. The method of claim 29 wherein there is provided a manifold between the electric fan and the one or more hollow conduits and wherein the method entails directing the air-fragrance mixture into the manifold and from the manifold into and through the one or more hollow conduits.
